(19) United States
(12) Patent Application Publication
(10) Pub. No.: US 2024/0074363 A1
(43) Pub. Date: Mar. 7, 2024

(54) PROCESS FOR SEEDING A SOLID LIGNOCELLULOSIC MATERIAL WITH A FUNGAL BIOMASS

(71) Applicants: INSTITUT NATIONAL POLYTECHNIQUE DE TOULOUSE, Toulouse (FR); INSTITUT NATIONAL DE RECHERCHE POUR L'AGRICULTURE L'ALIMENTATION ET L'ENVIRONNEMENT, PARIS (FR); UNIVERSITÉ D’AIX-MARSEILLE, MARSEILLE (FR)

(72) Inventors: Virginie VANDENBOSSCHE, SAINTE-FOY-D’AIGREFEUILLE (FR); Sana RAOUCHE, MARSEILLE (FR); Isabelle GIMBERT, MARSEILLE (FR); Christine DELGADO RAYNAUD, TOULOUSE (FR); William TAPIA, TOULOUSE (FR)

(21) Appl. No.: 18/282,514

(22) PCT Filed: Mar. 15, 2022
(86) PCT No.: PCT/EP2022/056751

§ 371 Date: Sep. 15, 2023

(30) Foreign Application Priority Data
Mar. 16, 2021 (FR) FR2102617

(51) Int. Cl. A01G 18/20; C08L 1/02; C08L 97/02

(52) U.S. Cl. CPC A01G 18/20; C08L 1/02; C08L 97/02

(57) ABSTRACT

The invention relates to a process for preparing a solid lignocellulosic material (1), referred to as a composite material, seeded with at least one organism (2), referred to as a filamentous fungus, which is a mycelium-forming multicellular eukaryote, in which:
at least one solid lignocellulosic material (3) impregnated with an aqueous composition (5) is subjected to a treatment (6), known as a thermomechanical treatment, in which
   said at least one impregnated lignocellulosic material (3) is subjected to a succession of mechanical compression, expansion and shearing phases by blending at least one solid lignocellulosic material (4) of said at least one impregnated lignocellulosic material (3), in contact with the aqueous composition (5);
   said at least one impregnated lignocellulosic material (3) is brought to a temperature above 50° C.;
      whereby a composition (7), referred to as a hydrated composition, comprising a solid lignocellulosic material (8), referred to as hydrated lignocellulosic material, the specific surface area and moisture content of which are increased relative to the specific surface area and moisture content of said at least one starting lignocellulosic material (4), is formed, said hydrated lignocellulosic material (8) being suitable for being colonized by said at least one filamentous fungus (2); and then
a composition, known as a fungal composition (9), comprising said filamentous fungus (2) is added to said hydrated composition (7) during blending;
   in which process the successive steps are performed continuously in at least one twin-screw extruder (10).

The invention relates to a process for seeding a solid lignocellulosic material with at least one filamentous fungus for the purpose of its development. The invention also relates to a process for manufacturing a solid object in which such a solid lignocellulosic material seeded with at least one filamentous fungus is used, and to a solid object made of composite material obtained via such a process.

Such a process for seeding at least one filamentous fungus in a solid lignocellulosic material for the purpose of its development finds its applications in the field of manufacturing solid objects made of composite materials obtained essentially—notably entirely—from natural biological resources—notably natural plant resources—which are inexpensive and renewable. Composite materials of this kind are thus inexpensive, taking into account the upgrading of waste materials, and constitute an alternative to the use of non-renewable fossil resources.

Such a process is of most particular interest in the more general field of the upgrading of plant resources, for instance plant waste—in particular plant waste from agriculture or the agrifood industry-, via which such plant waste is transformed into composite materials which are no longer waste.

Throughout the text:
the term “lignocellulosic material” denotes any natural material comprising at least one cellulose, at least one hemicellulose and/or at least one lignin;
the term “filamentous fungus” denotes any multicellular eukaryotic organism whose vegetative apparatus is in the form of filaments or hyphae, giving the vegetative apparatus a cottony appearance when observed visually on a macroscopic scale;
the term “mycelium” denotes all the filaments—branched or unbranched—forming the vegetative apparatus of such filamentous fungi, and;
the term “seeding” denotes the intimate mixing of the solid lignocellulosic material and the filamentous fungus/fungi in contact with the lignocellulosic fibres of the solid lignocellulosic material under conditions suitable for allowing the subsequent growth of the filamentous fungus/fungi in said composite material.

The review articles Eugene Soh et al., 2020, Materials and Design, 195, 109058 and Mitchell Jones et al., 2020, Materials and Design, 187, 108397 disclose composite construction materials formed from fungal mycelium.

Also as disclosed in WO 2019/226823 is a process for making a product formed from a mycelial biomaterial, in which aspen chips are mixed with an inoculum of Ganoderma lucidum so as to form a pourable composition. WO 2019/226823 states that the mixing may be performed in a continuous screw mixer and that the chips may be subjected to a subsequent sterilization step. In the process in WO 2019/226823, the mixture is placed in a first chamber, then compacted in this first chamber in which it is subjected to aeration for a time and temperature sufficient to allow the mycelium to grow and cover the chips. During a subsequent step, the chips are removed from the first chamber, mixed with nutrients and then poured into a second chamber having the desired shape of the mycelial biomaterial product.

The process described in WO 2019/226823 is complex to perform and requires numerous manipulations. In practice, it is not suitable for industrialization. The process of WO 2019/226823 is limited to the treatment of wood chips and does not allow the treatment of any type of lignocellulosic material, irrespective of its form, notably any type of fibrous lignocellulosic material. In particular, it does not allow the processing of any type of lignocellulosic material, for instance plant waste from agriculture or from the agrifood industry.

The invention is directed towards overcoming the drawbacks mentioned previously by proposing a process for seeding a solid lignocellulosic material with a fungal biomass, which is capable of being performed on an industrial scale.

The invention is directed in particular towards proposing such a process which is quick to perform.

Thus, the invention is directed towards such a process which allows such a solid lignocellulosic material seeded with a fungal biomass to be formed rapidly, for instance in less than an hour—notably in a time of the order of a few minutes.

The invention is directed towards such a process which allows the continuous formation of such a solid lignocellulosic material seeded with a fungal biomass.

The invention is also directed towards such a process which allows the continuous formation of such a solid lignocellulosic material seeded with at least one filamentous fungus, in which the subsequent development of the filamentous fungus/fungi is favoured via said process.

In particular, the invention is directed towards such a process which does not require the supply of nutrients for the growth of the filamentous fungus/fungi.

The invention is also directed towards such a process which allows continuous formation of such a solid lignocellulosic material seeded with a fungal biomass, with a particle size of said seeded solid lignocellulosic material which is controlled and chosen as a function of the desired use of said seeded solid lignocellulosic material.

The invention also is also directed towards proposing such a process allowing the upgrading of plant resources, which are no longer waste, and which are capable of being upgraded as raw material for the manufacture of objects made of composite material free from constituents of fossil origin.

In particular, the invention is directed in particular towards proposing such a process for treating a solid lignocellulosic material which is available in large amounts.

Another object of the invention is to propose a process for manufacturing a composite material from such a solid lignocellulosic material seeded with a fungal biomass.

The invention is also directed towards achieving these objects at a lower cost, by proposing such a process for preparing such a composite material, which is easy to perform.

To this end, the invention relates to a process for preparing a solid lignocellulosic material, referred to as a composite material, seeded with at least one organism, referred to as a filamentous fungus, which is a mycelium-forming multicellular eukaryote, in which:
at least one solid lignocellulosic material impregnated with an aqueous composition is subjected to a treatment, known as a thermomechanical treatment, in which
   said at least one impregnated solid lignocellulosic material is subjected to a succession of mechanical compression, expansion and shearing phases by blending at least one solid lignocellulosic material of said at least one impregnated lignocellulosic material, in contact with the aqueous composition;
   said at least one impregnated solid lignocellulosic material is brought to a temperature above 50° C.;
      whereby a composition, referred to as a hydrated composition, comprising a solid lignocellulosic material, referred to as hydrated lignocellulosic material, the specific surface area and moisture content of which are increased relative to the specific surface area and moisture content of said at least one starting lignocellulosic material, is formed as a result of this blending and this heating, said hydrated composition being suitable for being colonized by said at least one filamentous fungus; and then
a composition, referred to as a fungal composition, comprising said filamentous fungus is added to said hydrated composition undergoing blending under blending conditions suitable for preserving the viability of at least one such filamentous fungus and for allowing subsequent development of said filamentous fungus in said composite material;
   in which process the successive steps are performed continuously in at least one twin-screw extruder between an upstream inlet of said at least one twin-screw extruder, via which said at least one solid lignocellulosic material is introduced, and a downstream outlet of said at least one twin-screw extruder, via which said composite material is discharged.

In a process according to the invention, said at least one impregnated solid lignocellulosic material is subjected to said thermomechanical treatment in a twin-screw extruder and under conditions that are suitable for allowing defibration by at least partial deaggregation of the lignocellulosic fibres of the solid lignocellulosic material by mechanical and thermal means and by de-structuring of these lignocellulosic fibres, followed by the addition of said fungal composition, comprising at least one filamentous fungus that is capable of growing within said at least partially deaggregated lignocellulosic material—and with a specific surface area that is increased relative to the specific surface area of the starting lignocellulosic material—of said hydrated composition and of forming a cohesive binder for the lignocellulosic fibres of said composite material due to this growth, notably by producing mycelial hyphae.

In a process according to the invention, said thermomechanical treatment is a treatment for at least partial sterilization of the impregnated solid lignocellulosic material. The process according to the invention is a process for preparing said composite material in which the solid lignocellulosic material forms a support that is capable of allowing the development of said filamentous fungus in said composite material.

The inventors have determined that the twin-screw extruder allows, by using a valve for introducing said fungal composition into the internal volume of the twin-screw extruder provided in the barrel by the screws of the twin-screw extruder, a gradual and perfectly controlled addition—notably at a constant or variable rate—of said fungal composition to said hydrated composition as it is conveyed through the twin-screw extruder. Such a gradual and controlled addition allows a controlled volume of said fungal composition to be added to a small volume of said hydrated composition undergoing conveying and blending flowing opposite the entry path of said fungal composition into the internal volume of the twin-screw extruder. Thus, the ratio of the volume of said hydrated composition receiving said fungal composition to the volume of said fungal composition supplied to the corresponding volume of said hydrated composition is small and in any case smaller than the ratio of the total volume of a hydrated composition prepared in a reactor receiving said fungal composition to the volume of said fungal composition supplied to the corresponding total volume of said hydrated composition. Thus, the mixing of said fungal composition into said hydrated composition is performed more efficiently than the mixing performed by adding said fungal composition into the total volume of said hydrated composition, such as is performed during “batch” mixing in a mixing reactor.

Thus, the addition of said fungal composition to said hydrated composition under conditions of conveying and blending of said hydrated composition in the twin-screw extruder allows:
optimum dispersion of said fungal composition in said hydrated composition and distribution of the filamentous fungus/fungi of said fungal composition in contact with the at least partially deaggregated lignocellulose fibres of said hydrated composition; and
intimate mixing of said fungal composition and the at least partly deaggregated lignocellulose fibres of said hydrated composition;
   while at the same time preserving—at least partly—the viability of the filamentous fungus/fungi of said fungal composition during this intimate mixing. Thus, as a result of this preservation of the viability of the filamentous fungus/fungi in said composite material formed as a result of this mixing, the subsequent development of the filamentous fungus/fungi within said composite material is promoted.

During said thermomechanical treatment, the solid lignocellulosic material of said impregnated solid lignocellulosic material undergoes at least partial destructuring by blending and/or grinding and/or shearing and/or compressing and/or expanding said solid lignocellulosic material combined with heat treatment at a temperature above 50° C. leading to a reduction in the cohesion and at least partial dissociation of the lignocellulosic fibres making up the solid lignocellulosic material. Such a thermomechanical treatment allows the particle size of said solid lignocellulosic material to be reduced to suit the end use of said composite material formed. Such a reduction in particle size allows a more homogeneous distribution of said at least one filamentous fungus in said composite material to be promoted, even for large volumes of said prepared composite material.

Advantageously, the impregnation of said at least one solid lignocellulosic material with an aqueous composition prior to said thermomechanical treatment allows uncontrolled heating of said impregnated lignocellulosic material to be avoided and allows its subsequent defibration to be facilitated.

Said hydrated composition is a composition which is substantially free of free water liable to flow spontaneously from said hydrated composition. The water in said hydrated composition is associated with said solid lignocellulosic material of increased specific surface area and moisture content relative to the specific surface area and moisture content of said at least one lignocellulosic starting material. The amount of water associated with said solid lignocellulosic material of increased specific surface area and increased moisture content depends on the solid lignocellulosic material itself. In general, said impregnated solid lignocellulosic material is formed in such a way that the mass of solids of said impregnated solid lignocellulosic material to the mass of said impregnated solid lignocellulosic material is between 30% and 60%, notably between 40% and 50%.

The inventors have observed that the successive mechanical compression, expansion and shearing phases to which said solid lignocellulosic material is subjected in the twin-screw extruder during said thermomechanical treatment not only allow the formation of a deaggregated and hydrated lignocellulosic material, suitable for supporting the subsequent development of said filamentous fungus, but also allow at least partial inactivation of at least part of the endogenous microbial flora of said solid lignocellulosic material. This at least partial inactivation of this endogenous flora allows the subsequent development of said filamentous fungus in contact with the lignocellulosic fibres of said hydrated lignocellulosic material. However, the inventors also defined conditions for conveying said hydrated lignocellulosic material into the twin-screw extruder, making it possible to achieve intimate mixing of the filamentous fungus/fungi at the heart of the lignocellulosic fibres of said hydrated lignocellulosic material, forming said composite material which is suitable for being able to be subjected to a development step, notably fermentation, on a solid medium of the filamentous fungus/fungi in contact with the lignocellulosic fibres of said hydrated lignocellulosic material.

The effects of blending and/or grinding and/or shearing and/or compression and/or expansion are obtained here through sustained movement of at least one rigid mechanical member within and in contact with said solid lignocellulosic material of said impregnated solid lignocellulosic material. This movement is said to be maintained due to the fact that it continues during said thermomechanical treatment. Said thermomechanical treatment may thus be continuous or discontinuous.

Throughout the text hereinbelow, the term “twin-screw extruder” denotes a blending device comprising two co-penetrating screws with direct or reverse pitch driven in rotation in synchronism inside a tubular barrel having a bilobal shape in cross-section. The sleeve and the co-penetrating screws may be made of any industrial alloy suitable for the operating conditions, in particular in terms of abrasion and corrosion. Each of the co-penetrating screws may be formed from screw sections extending axially and successively on a splined axis and axially providing successive zones for treating the solid lignocellulosic material between an inlet or loading zone of the twin-screw extruder and an outlet or discharge zone.

One advantage of using a twin-screw extruder is the speed of processing. Specifically, under the conditions for performing the process according to the invention, a few minutes to a few tens of minutes, depending on the dimensions of the twin-screw extruder, are sufficient to prepare said composite material. The screw profile—as defined by the sequence, shape and pitch of the constituent elements of the screws of the twin-screw extruder—and the speed of rotation of the screws in the barrel are chosen to obtain a blending and/or grinding and/or shearing and/or compression and/or expansion effect of said solid lignocellulosic material and to allow a residence time of said impregnated solid lignocellulosic material in the twin-screw extruder, which are suitable for allowing at least partial deaggregation of the lignocellulosic fibres of said solid lignocellulosic material.

For the implementation of a process according to the invention, such a twin-screw extruder has the advantage of allowing all the steps to be automated and the treatment to be performed in a single continuous operation.

The use of a twin-screw extruder advantageously allows precise control of a large number of operating parameters of a process according to the invention (processing temperature, mode and force of compression, expansion and shearing, processing time, etc.). By modifying certain structural characteristics and/or certain operating characteristics of the twin-screw extruder, the operator can influence the process parameters.

As an example of structural characteristics of a twin-screw extruder on which an operator may intervene, and which are generally determined and fixed before it is started up (and which are not normally modifiable during its operation), mention may be made in particular of the profile of the screws, which depends essentially on the type of screw used. The screws may be conveying screws, blending screws (such as monolobal or bilobal blending elements) or shear stress screws (such as counter-thread screws). The screws may differ in the shape of their thread (which may be trapezoidal, conjugate, single or double, for instance) and in the size of their screw pitch. Each of these screws may also have different sections (or segments) which may differ from each other in terms of the thread shape and/or the screw pitch. Optionally, some of these sections making up these screws may also correspond to monolobal or bilobal blending elements.

Among these operating characteristics of a twin-screw extruder used in a process according to the invention, on which an operator may intervene at any time (both when this equipment is stopped and when it is running), mention may be made notably of:
the screw rotation speed;
the barrel temperature. Particular temperatures may be set in different sections extending longitudinally in this barrel.

The wide choice in the structural and operating characteristics of such a twin-screw extruder allows great freedom in adjusting the conditions for performing a process according to the invention, and in particular in defining optimum conditions (for example the temperature, shear force, processing time, liquid/solid ratio) specific to each of the solid lignocellulosic starting materials chosen. Advantageously, the screws of a twin-screw extruder used in the context of the invention may comprise at least two sections which differ in their screw profile.

These structural characteristics of the screws of the twin-screw extruder are suitable for allowing not only conveying of the solid lignocellulosic material of said impregnated solid lignocellulosic material longitudinally in the barrel of the twin-screw extruder, but also for forming, during this conveying, zones of compression and/or expansion and/or blending and/or grinding and/or shearing and/or swelling by hydration of the solid lignocellulosic material of said impregnated solid lignocellulosic material and colonization of the deaggregated lignocellulosic fibres of said hydrated composition by the filamentous fungus/fungi.

According to certain advantageous embodiments, the process is performed continuously by means of a single twin-screw extruder. Such a single twin-screw extruder allows said impregnated solid lignocellulosic material flowing continuously from upstream to downstream of the twin-screw extruder to be subjected to said thermomechanical treatment in an upstream part of the twin-screw extruder, then in a downstream part of the twin-screw extruder, to mixing:
of said hydrated composition conveyed by the twin-screw extruder and formed due to said thermomechanical treatment and substantially free from any endogenous flora of said solid lignocellulosic starting material; and
of said fungal composition introduced into the twin-screw extruder via an introduction valve;
   under mixing conditions suitable for at least partially preserving the viability of said filamentous fungus/fungi.

In certain embodiments, said thermomechanical treatment of the process according to the invention is a treatment for inactivating at least part of the endogenous microbial flora of said at least one solid lignocellulosic material. In these embodiments, the heating temperature and the heating time of said impregnated solid lignocellulosic material in the twin-screw extruder are chosen so as to inactivate at least part—notably a major part, in particular substantially all—of the endogenous microbial flora of said solid lignocellulosic material. Such inactivation of the endogenous microbial flora initially present in said solid lignocellulosic material promotes the implantation and subsequent development of the filamentous fungus/fungi by limiting the risks of trophic competition with the endogenous flora of said solid lignocellulosic material.

In these advantageous embodiments, the thermomechanical treatment and the addition of said fungal composition to said hydrated composition—and, where appropriate, a cooling of said hydrated composition—are performed successively in the same twin-screw extruder at a temperature suitable for preserving at least some—notably all—of the viability of said filamentous fungus/fungi and for allowing subsequent development of said filamentous fungus/fungi in said composite material. To do this, the twin-screw extruder has:
an upstream zone for thermomechanical treatment of said impregnated solid lignocellulosic material;
where appropriate, an intermediate zone for cooling said impregnated solid lignocellulosic material; and
a downstream zone for adding said fungal composition to said impregnated solid lignocellulosic material—which may have been cooled—and for mixing said fungal composition with said impregnated solid lignocellulosic material.

In other embodiments, the thermomechanical treatment and the addition of said fungal composition into said hydrated composition are performed successively in different twin-screw extruders.

According to certain embodiments, the heating temperature of said at least one impregnated solid lignocellulosic material during the thermomechanical treatment is between 50° C. and 180° C., preferably between 130° C. and 150° C.

According to certain embodiments, said hydrated composition is cooled to a temperature below 50° C., notably between 5° C. and 50° C., prior to the addition of said fungal composition. According to certain embodiments, said hydrated composition is cooled continuously in the twin-screw extruder, by conveying said hydrated composition to be cooled into at least one section of the twin-screw extruder provided with a barrel maintained at the cooling temperature. According to certain embodiments, said hydrated composition is cooled continuously in the twin-screw extruder by conveying said hydrated composition to be cooled in at least one section of the twin-screw extruder equipped only with conveying screws, i.e. screws without alternate compression and expansion members. Such combined and inter-penetrating conveying screws allow said hydrated composition to be conveyed through the extruder and promote heat exchange with the barrel maintained at the cooling temperature.

According to certain embodiments, said at least one impregnated solid lignocellulosic material comprises an amount of said at least one solid lignocellulosic material such that the ratio of the mass of dry matter of said at least one solid lignocellulosic material to the mass of said at least one impregnated solid lignocellulosic material is between 30% and 60%, notably between 40% and 50%. The ratio of the mass of dry matter of said solid lignocellulosic material of the impregnated solid lignocellulosic material to the total mass of said impregnated solid lignocellulosic material is between 30% and 60%.

According to certain embodiments of a process according to the invention, said impregnated solid lignocellulosic material is formed in the twin-screw extruder by introducing a flow of aqueous composition into the flow of lignocellulosic material which is being conveyed in the twin-screw extruder, so as to form an impregnated solid lignocellulosic material comprising an amount of solid lignocellulosic material such that the ratio of the mass of dry matter of said at least one solid lignocellulosic material to the mass of said impregnated solid lignocellulosic material is between 30% and 60%, notably between 40% and 50%.

The mass of such dry matter is determined via a method known per se to those skilled in the art, via which said matter is weighed after having been exposed to drying at a temperature of the order of 103° C. for a time required to obtain a substantially constant mass, this substantially constant mass being representative of the mass of the dry matter of said solid lignocellulosic material.

According to certain embodiments, the amount of aqueous composition in said at least one impregnated solid lignocellulosic material—notably the amount of water in said at least one impregnated solid lignocellulosic material—is adjusted so that said composite material is free of free aqueous composition liable to flow spontaneously from said composite material. According to certain embodiments, the amount of aqueous composition—notably the amount of water—in said at least one impregnated solid lignocellulosic material is adjusted so that said composite material has an optimized moisture content to allow optimum growth of the filamentous fungus/fungi. According to certain embodiments, said composite material comprises an amount of said hydrated lignocellulosic material (of increased specific surface area and moisture content relative to the specific surface area and moisture content of said at least one starting lignocellulosic material) such that the ratio of the mass of dry matter of said hydrated lignocellulosic material to the mass of said composite material is between 10% and 30%, notably between 15% and 30%.

According to certain particular embodiments, said hydrated lignocellulosic material has a particle size smaller than that of said at least one starting solid lignocellulosic material and smaller than that of said at least one impregnated solid lignocellulosic material. This particle size may vary according to the choice of solid lignocellulosic starting material and according to the conditions of implementation of said thermomechanical treatment, notably according to the profile of the twin-screw extruder. As a guide, according to certain embodiments of a process according to the invention, the lignocellulosic fibres of said hydrated composition have an average larger dimension (notably a length) of between 10⁻³ m and 10⁻² m and a smaller dimension (notably a diameter) orthogonal to the larger dimension of less than 2×10⁻³ m.

Any type of filamentous fungus may be used provided that it is of the mycelium-producing type. According to certain embodiments, said filamentous fungus is chosen from the group formed by organisms in the Basidiomycota phylum. According to certain particular embodiments, said filamentous fungus is chosen from the group formed by Grammothele fuhligo, Pleurotus citrinopileatus (yellow oyster mushroom), Lentinula edodes (or oak or shiitake or shiitake oyster mushroom), Pleurotus ostreatus (oyster mushroom or grey oyster mushroom), Pleurotus pulmonarius (lung oyster mushroom), Pleurotus columbinus, oyster mushroom hybrids, Ganoderma resinaceum (softwood Ganoderma), Agrocybe brasihensis, Flammulina velutipes, Hypholoma capnoides, Hypholoma sublaterium, Morchella angusticeps, Macrolepiota procera, Coprinus comatus, Agaricus arvensis, Ganoderma tsugae, Ganoderma lucidum and Inonotus obliquus. Such filamentous fungi growing in contact with deaggregated lignocellulosic fibres in said composite material produce biological material forming a cohesive binder in said composite material by filling spaces vacated by the lignocellulosic fibres. Such biological material may comprise structural proteins constituting the mycelium of the filamentous fungus/fungi, and/or excreted proteins and/or chitin.

According to certain embodiments, said at least one impregnated solid lignocellulosic material is prepared prior to its introduction into the twin-screw extruder, by adding the aqueous composition to said at least one solid lignocellulosic material.

According to certain embodiments, said at least one solid lignocellulosic material comprises:
a mass proportion of celluloses, expressed as dry weight of celluloses and as dry weight of said at least one solid lignocellulosic material (i.e. by the ratio of the mass of cellulose dry matter to the mass of dry matter of said at least one dry lignocellulosic material) of between 20% and 99%, notably between 20% and 98%, in particular between 20% and 90%, preferably between 30% and 60%;
a mass proportion of hemicelluloses, expressed as dry weight of hemicelluloses and as dry weight of said at least one solid lignocellulosic material (i.e. by the ratio of the mass of dry matter of the hemicelluloses to the mass of dry matter of said at least one solid lignocellulosic material) of between 10% and 50%, notably between 10% and 35%;
a mass proportion of lignins, expressed as dry weight of lignins and as dry weight of said at least one solid lignocellulosic material (i.e. by the ratio of the mass of dry matter of lignins to the mass of dry matter of said at least one solid lignocellulosic material) of between 0.1% and 35%, notably between 0.1% and 30%.

Any solid lignocellulosic material may be used in a process according to the invention, including cotton.

According to certain embodiments, at least one solid lignocellulosic material is chosen from the group consisting of all or part of a herbaceous plant—in particular a cereal (for instance wheat, barley, rice, oats, notably), a cereal straw, the stalks of a cultivated plant (for instance sorghum, corn, sugar cane, etc.), all or part of a woody plant (bark, wood chips), a waste product from a plant resulting from the upgrading of said plant (shives, oilseed cake, notably) and all or part of a plant producing vegetable fibres such as sisal, flax, coconut, hemp, jute, ramie, cotton, nettle, notably.

According to certain embodiments, said fungal composition is added to said hydrated composition maintained at a temperature of between 10° C. and 30° C., notably between 20° C. and 30° C. In these embodiments, said hydrated composition is cooled as it is conveyed through the twin-screw extruder so as to reach this temperature of between 10° C. and 30° C. The temperature of said hydrated composition is adjusted by cooling at least one longitudinal section of the barrel of the twin-screw extruder.

According to certain embodiments, said thermomechanical treatment is performed continuously in at least one twin-screw extruder comprising, from upstream to downstream, a succession of rotary screw sections coupled to a tubular barrel of the twin-screw extruder, suitable for said at least one impregnated solid lignocellulosic material to be subjected, during its conveying from upstream to downstream in the twin-screw extruder, to increasing compression, shear and expansion stresses.

According to certain embodiments, the succession of rotating screw sections coupled to the tubular barrel of the twin-screw extruder comprises at least one conveying screw and at least one stressing screw—such as screws of the bilobal blending type mounted at 90° or −45°, or of the perforated counter-thread type or screws with a wider pitch than the conveying screws placed upstream.

In certain embodiments, said thermomechanical treatment is performed continuously in at least one twin-screw extruder having, from upstream to downstream, in this order:
at least one section equipped with a conveying screw chosen from the group formed by single-threaded conjugate screws of C1F type, double-threaded conjugate screws of C2F type, double-threaded trapezoidal screws of T2F type, single-threaded trapezoidal screws of T1F type and variants thereof; and then
at least one section equipped with a stress screw chosen from the group consisting of monolobal screws mounted at +45°, bilobal screws mounted at +45° (corresponding to an angle offset of +45° of the elements relative to each other), monolobal screws mounted at +90°, bilobal screws mounted at +90° (corresponding to an angle offset of +90° of the elements relative to each other), monolobal screws mounted at −45°, bilobal screws mounted at −45° (corresponding to an angle offset of −45° of the elements relative to each other) and inverted screws, known as “counter-threads”, of the openwork CF2C type; and then
at least one section equipped with conveying screws chosen from the group formed by single-threaded conjugate screws of the C1F type, double-threaded conjugate screws of the C2F type, single-threaded trapezoidal screws of the T1F type, double-threaded trapezoidal screws of the T2F type and variants thereof.

In certain embodiments, said fungal composition is a liquid composition. Needless to say, in these embodiments, said liquid fungal composition is added to said hydrated composition so that said composite material has an optimized moisture content suitable for allowing optimal growth of said filamentous fungus/fungi. In particular, said liquid fungal composition is added to said hydrated composition so that said composite material comprises an amount of said hydrated lignocellulosic material (of increased specific surface area and moisture content relative to the specific surface area and moisture content of said at least one starting lignocellulosic material) such that the ratio of the mass of dry matter of said hydrated lignocellulosic material to the mass of said composite material is between 10% and 30%, notably between 15% and 30%. In these embodiments, the mixing of said liquid fungal composition and said hydrated composition is performed in at least one twin-screw extruder section equipped with screws of a type of conjugate and inter-penetrating conveying screws. Such a type of conveying screw allows effective mixing and effective impregnation of said fungal composition in said hydrated lignocellulosic material to form said composite material.

In some of these embodiments, said fungal composition is a solid composition. In these embodiments, the mixing of said solid fungal composition and of said blended dispersion is performed in at least one twin-screw extruder section equipped with
conveying screws chosen from the group formed by single-threaded conjugate screws of C1F type, double-threaded conjugate screws of C2F type, double-threaded trapezoidal screws of T2F type, single-threaded trapezoidal screws of T1F type and variants thereof; and/or
stressing screws of the monolobal blender type or the bilobal blender type (BB+45° or MAL0 +45°, whose monolobes or bilobes are respectively oriented perpendicular to the splined shafts and are offset relative to each other by an angle of +45°; and/or
stressing screws of the monolobal blender type or the bilobal blender type (BB 90° or MAL0 90°), whose monolobes or bilobes are respectively oriented perpendicular to the splined shafts and are offset from each other by an angle of 90°.

The invention covers a composite material obtained via a process according to the invention.

However, the present invention also covers a solid lignocellulosic material, referred to as a composite material, comprising lignocellulosic fibres and at least one organism, referred to as a filamentous fungus, which is a mycelium-forming multicellular eukaryote, said composite material being predominantly in the form of particles of generally elongate shape and having a largest dimension (a length) greater than each of the two dimensions (width and thickness) orthogonal to the largest dimension and orthogonal to each other, the ratio of the largest dimension (length) to each of the dimensions orthogonal to the largest dimension (width and/or thickness) being greater than 2, notably greater than 4, preferably greater than 6, even more preferentially greater than 10.

The invention also covers any use of such a composite material obtained via a preparation process according to the invention.

In particular, the invention relates to the use of said composite material—whether obtained via a preparation process according to the invention or not—for the purpose of at least partial—notably total—digestion of a solid lignocellulosic material which may notably be decomposed by said developing filamentous fungus/fungi—in particular prior to a step of enzymatic hydrolysis of the cellulose of the decomposed solid lignocellulosic material and its conversion into fermentable sugars for the production of ethanol. The invention thus relates to a process according to the invention for preparing a composite material for the purpose of at least partial—notably total—digestion of said solid lignocellulosic material.

The invention also covers a process for manufacturing a solid object in which a process according to the invention for preparing said composite material is performed. The invention also covers such a process for manufacturing a solid object in which a composite material obtained via a process according to the invention is chosen, and said composite material is then formed and said composite material thus formed is subjected to a fermentation step, known as fermentation in a solid medium, and to the development of said at least one filamentous fungus in said composite material. Said fermentation in a solid medium may be an anaerobic or aerobic fermentation. The fermentation in a solid medium and the development of the filamentous fungus/fungi take place in contact with the hydrated lignocellulosic material of said composite material in the virtual absence of free water, said hydrated lignocellulosic material acting as a support for this fermentation and this development, but also, where appropriate, as a nutrient source for the filamentous fungus/fungi.

According to certain embodiments of a process according to the invention for manufacturing a solid object, the solid object manufactured being a low-density moulded object:
said composite material obtained via a process for preparing said composite material according to the invention is formed, in particular by moulding; and then
said formed composite material is subjected to a step of fermentation, known as fermentation in a solid medium, and of development of said at least one filamentous fungus in said composite material, whereby a mycelium-enriched material is formed; and then
the mycelium-enriched material is dried so as to form the moulded solid object consisting of a low-density composite material. In these embodiments, the low-density composite material may have a density of less than 0.1 g/cm³.

In other embodiments of a process according to the invention for manufacturing a solid object, the solid object manufactured being an object of density greater than 0.1 g/cm³, notably of density between 0.6 g/cm³ and 1.4 g/cm³, in particular between 0.6 g/cm³ and 1.0 g/cm³, and greater than the density of the mycelium-enriched material forming a solid object obtained by simple moulding:
said composite material is subjected to a fermentation step, known as fermentation in a solid medium, and to the development of said at least one filamentous fungus in said composite material and in contact with said hydrated lignocellulosic material, whereby a mycelium-enriched material is formed; and then
said mycelium-rich material is subjected to a step of forming by thermocompression, where appropriate after drying and/or grinding of said mycelium-enriched material, so as to form the solid object with a density greater than 0.1 g/cm³. In these embodiments, the material forming the solid object has a density of greater than 0.1 g/cm³, notably between 0.6 g/cm³ and 1.4 g/cm³, in particular between 0.6 g/cm³ and 1.0 g/cm³.

According to certain embodiments, the manufacturing process does not comprise any addition to said composite material of a nutrient composition to aid the development of said at least one filamentous fungus/fungi. However, according to other embodiments, there is nothing to prevent the addition, during the formation of said composite material or into said composite material, of a nutrient composition to aid the development of said at least one filamentous fungus.

The invention also relates to a process for preparing a solid lignocellulosic material, referred to as a composite material, seeded with at least one filamentous fungus, and to a process for manufacturing a solid object, characterized in combination by all or some of the characteristics mentioned above or hereinbelow. Irrespective of the formal presentation that is given thereof, unless explicitly indicated otherwise, the various characteristics mentioned hereinabove or hereinbelow should not be considered as being strictly or inextricably linked together, the invention being able to concern only one of these structural or functional characteristics, or only a portion of these structural or functional characteristics, or only a portion of one of these structural or functional characteristics, or else any group, combination or juxtaposition of all or a portion of these structural or functional characteristics.

Other aims, features and advantages of the invention will become apparent on reading the following examples, which are given solely by way of illustration and are non-limiting illustrations of certain possible embodiments of the invention, and from the following description of certain possible embodiments of the invention with reference to the appended drawings in which:

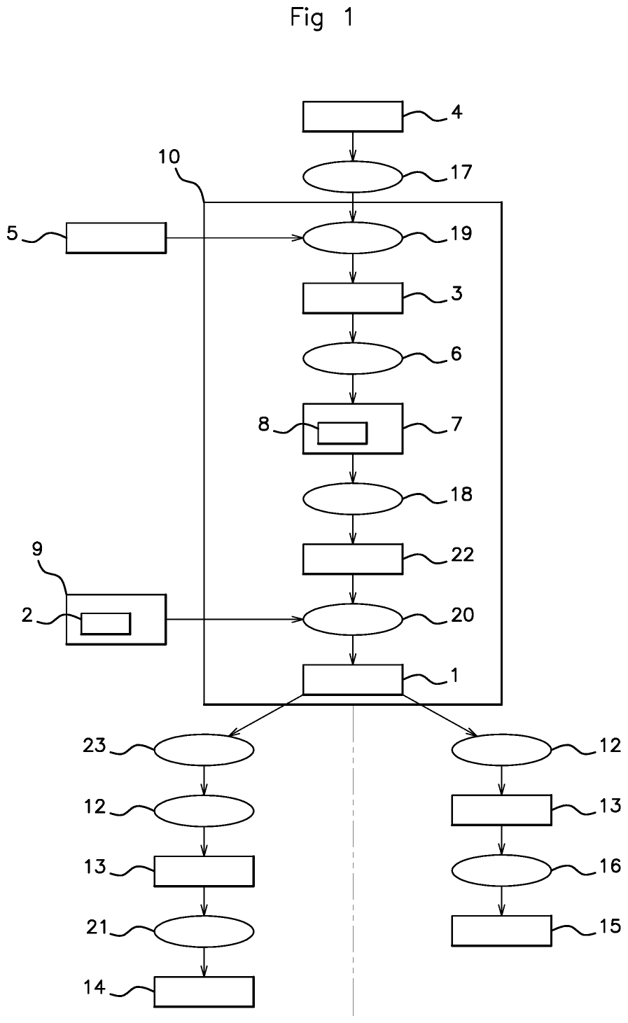

FIG. 1 is an overview diagram of a variant of a process according to the invention;

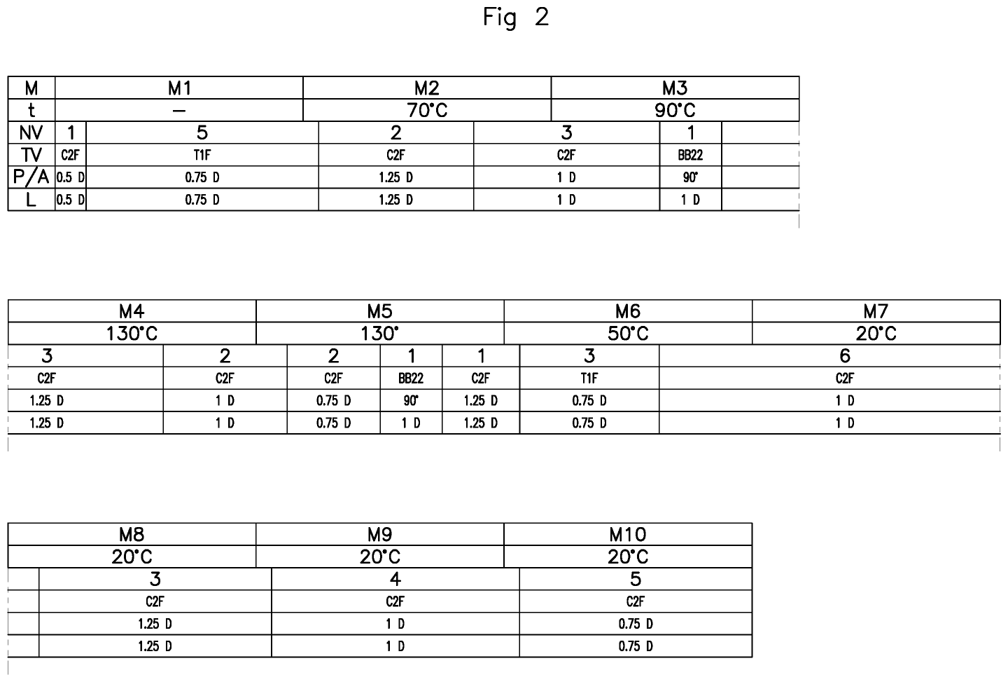

FIG. 2 is a block diagram of an example of a twin-screw extruder which may be used for performing a first variant of a process according to the invention; and

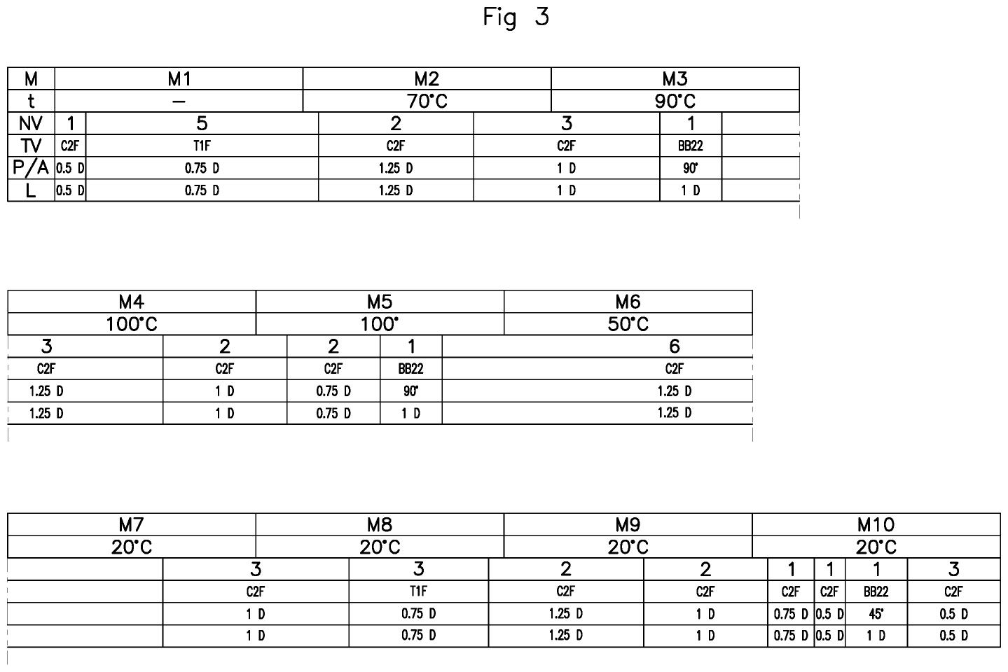

FIG. 3 is a block diagram of an example of a twin-screw extruder which may be used for performing a second variant of a process according to the invention.

In a process for preparing a solid lignocellulosic material, known as a composite material 1, seeded with at least one filamentous fungus 2, a solid lignocellulosic material 4 composed of celluloses, hemicelluloses and lignin is chosen. In a process according to the invention, such a solid lignocellulosic material 4 may be a plant material from agriculture or forestry or growing in the wild. It may be all or part of an agricultural product. It may notably be a part of such agricultural production, considered as a waste product with respect to this agricultural production and which, due to its use in a process according to the invention, constitutes an upgrade of this agricultural production. Advantageously, the solid lignocellulosic material 4 is formed from a plant resource which is renewable.

In a process for preparing said composite material 1, a substantially continuous introduction 17 of a flow of the fragmented solid lignocellulosic material 4 is performed upstream of a twin-screw extruder 10 configured so as to be able to receive the solid lignocellulosic material 4 and to convey this solid lignocellulosic material 4 between the upstream inlet of the lignocellulosic material 4 into the twin-screw extruder 10 and a downstream outlet of said composite material 1 formed in the extruder 10. During the conveying of the solid lignocellulosic material 4 in the extruder 10, a flow of an aqueous composition 5—notably water—is fed 19 into the barrel of the extruder 10 so as to form a solid lignocellulosic material 3 impregnated with aqueous composition 5 as a result of the conveying of the solid lignocellulosic material 4 and its blending. The flow rate of aqueous composition 5 is adjusted according to the nature and composition of the solid lignocellulosic material 4, so that the ratio of the mass of dry matter of the solid lignocellulosic material 4 in said impregnated lignocellulosic material 3 is between 40% and 60%.

Said impregnated lignocellulosic material 3 is then subjected, as it is conveyed through the extruder 10, to a treatment known as a thermomechanical treatment 6, in which said impregnated lignocellulosic material 3 is subjected to a succession of mechanical compression, expansion and shearing phases by blending said at least one solid lignocellulosic material 4 in contact with the aqueous composition 5 and to heating to a temperature above 50° C., notably between 50° C. and 180° C. However, there is nothing to prevent said thermomechanical treatment 6 from being performed at a temperature above 180° C. but without risking burning the lignocellulosic material 4. The thermomechanical treatment 6 of said impregnated lignocellulosic material 3 is performed by heating the barrel(s) of the module(s) of the extruder 10 corresponding to the heating zone. Due to the heating and the succession of mechanical compression, expansion and shearing phases to which the solid lignocellulosic material 4 is subjected in the extruder 10, a hydrated composition 7 is thus formed in the extruder 10, comprising a hydrated lignocellulosic material 8 of increased specific surface area and moisture content relative to the specific surface area and moisture content of the lignocellulosic material 4 introduced into the extruder 10. The conditions of said thermomechanical treatment 6, notably compression/expansion and heating, are chosen so as to form a hydrated composition 7 substantially free of endogenous microbial flora of the starting solid lignocellulosic material 4. According to the invention, said thermomechanical treatment 6 allows said hydrated composition 7 to be formed in which said hydrated lignocellulosic material 8 is suitable for being subsequently colonized by a filamentous fungus 2.

In a process according to the invention for preparing a composite material 1, on conclusion of said thermomechanical treatment 6, said hydrated composition 7 is subjected to cooling 18 by continuing to convey said hydrated composition 7 through the barrels of successive modules, known as cooling modules, of the extruder 10 which are maintained at low temperature, notably at a temperature of between 10° C. and 30° C., corresponding to a zone of the extruder 10 for cooling said hot hydrated composition 7. There is nothing to prevent the maintenance of mechanical compression, expansion and shearing phases during cooling 18 likely to promote heat exchanges between said hydrated composition 7 and the barrel of the cooling modules.

The hydrated composition 22 thus cooled is conveyed from upstream to downstream of extruder 10 in an extruder 10 module provided with an inlet for a composition, known as fungal composition 9, comprising at least one filamentous fungus 2. In a process according to the invention, said fungal composition 9 is added to said cooled hydrated composition 22 undergoing blending, under blending conditions suitable for at least partially preserving the viability of the filamentous fungus/fungi 2. A material, referred to as composite material 1, is thus formed from a solid lignocellulosic material seeded with viable filamentous fungi 2, substantially uniformly distributed in the solid lignocellulosic material and capable of developing mycelium in contact with the solid lignocellulosic material and of colonizing it. Blending is maintained by conveying said composite material 1 in the extruder 10 so as to promote redistribution of the filamentous fungi 2 in contact with the lignocellulosic fibres of the lignocellulosic material. Said composite material 1 is continuously expelled from the extruder 10 at its downstream longitudinal end.

In a first variant of a use of said composite material 1 represented in FIG. 1, said composite material 1 is subjected to a treatment 23 for forming said composite material 1. This may involve spreading said composite material 1 on a support of predetermined shape or any other type of forming. The formed composite material is then placed under conditions that allow the filamentous fungus/fungi 2 in contact with the lignocellulosic fibres to develop by fermentation 12 on a solid medium. The development of the mycelium of the filamentous fungus/fungi 2 in contact with the lignocellulosic fibres allows the formation of a fungal binder ensuring the cohesion of a mycelium-enriched material 13. After a drying treatment 21—notably by hot drying—of the mycelium-enriched material 13, a low-density solid object 14 is formed.

In a second variant for the use of said composite material 1 represented in FIG. 1, said composite material 1 is placed under conditions suitable for allowing the development of filamentous fungus/fungi 2 in contact with the lignocellulosic fibres, by fermentation 12 on a solid medium. The development of the mycelium of the filamentous fungus/fungi 2 in contact with the lignocellulosic fibres allows the formation of a fungal binder ensuring the cohesion of a mycelium-enriched material 13. The mycelium-enriched material 13 is then formed by thermocompression, forming a solid object 15 with a density of greater than 0.1 g/cm³. There is nothing to prevent this solid object 15 from undergoing a drying step intended to stabilize the fungal binder and halt the development of the filamentous fungus/fungi 2. For example, the mycelium-enriched material 13 is subjected to a step of compression under a pressure of 785 kg/cm² so as to form a standardized specimen. The standardized specimen has a flexural strength value of about 19 MPa and a flexural modulus of elasticity of about 2200 MPa. By way of comparison, a standardized specimen obtained by compressing a solid lignocellulosic material which has undergone said thermomechanical treatment but which is not enriched in mycelium and subjected to the same compression step under 785 kg/cm², has a flexural strength value of about 7 MPa and a flexural modulus of elasticity of about 370 MPa. This compressed material not enriched with mycelium is a friable material, unlike the material forming the solid object 15 according to the invention.

According to the invention, notably according to these first and second variants, the development of the mycelium of the filamentous fungus/fungi 2 in contact with the lignocellulosic fibres is made possible due to the thermomechanical treatment 6 of the solid lignocellulosic material 3 and its sterilization, at least partial, due to this treatment 6 in the extruder 10. The inventors have observed that a solid lignocellulosic material which has not undergone said at least partially sterilizing thermomechanical treatment 6 does not allow the mycelium of the filamentous fungus/fungi 2 to develop. The inventors assume that the development of the natural flora of such solid lignocellulosic material opposes the development of the filamentous fungus/fungi 2.

### Example 1—Seeding of Shives with a Liquid Suspension of the Filamentous Fungus Grammothele fuligo

Solid Lignocellulosic Material

The solid lignocellulosic material chosen is hemp shives composed of 44% celluloses, 18% hemicelluloses and 28% lignin, formed of fragments of generally cylindrical shape with an average cross-sectional diameter of between 0.5 mm and 6.3 mm. In particular, 70% of the fibres in the hemp shives have a diameter of greater than 2 mm. An analysis, by successive sieving on sieves of decreasing calibrated mesh, of the mass proportion of each of the size categories of the fragments forming the shives is given in Table 1 hereinbelow, in which “d” represents the diameter of the fragments and “L” represents their length.

TABLE 1

| | | | | |
|---|---|---|---|---|
| Size category, mm | 8 > d > 4 | 4 > d > 2.4 | 2.4 > d > 1 | d < 1 mm |
| Mass proportion, % | 6.2 | 35.7 | 55.3 | 2.8 |
| Length, mm | 10 < L < 40 | 6 < L < 20 | 3 < L < 15 | L < 10 |

The dry matter content of the hemp shives at equilibrium in ambient atmospheric air is 87% (ratio of the constant mass of hemp shives maintained at a temperature of 103° C. to the mass of hemp shives at equilibrium in ambient atmospheric air at room temperature).

Twin-Screw Extruder

The twin-screw extruder chosen is a Clextral EV25 (Clextral SA, Firminy, France) comprising a fixed hollow barrel forming a longitudinal bore of bilobal shape in cross-section, and two identical parallel shafts each driven in rotation in one of the lobes of the bilobal barrel along the longitudinal axis of each of the two shafts and at an identical speed of rotation and in the same direction of rotation. The barrel is made up of 10 successive bilobal modules of the same size (each 100 mm long) integrally mounted linearly with respect to each other. Each rotating shaft is equipped longitudinally with a succession of screw sections integrally mounted so as to rotate with the shafts. Each screw has a maximum cross-sectional diameter of 25 mm, the screws mounted opposite each other on each of the two shafts being of the same length and of the co-penetrating type. These co-penetrating screws are dimensioned and suitable for cooperating with the bilobal bore of the barrel to subject the hemp shives to conveying in a generally longitudinal direction relative to the barrel and to mechanical shearing and mixing work by means of successive sequences of compression, shearing and expansion of the material in the extruder barrel. The rotational speed of each shaft and screw is 200 revolutions per minute (rpm). The particular configuration of the Clextral EV25 twin-screw extruder is described by way of example in FIG. 2.

The extruder barrel is represented schematically in FIG. 2 and extends over a total length of 1000 mm. In the schematic representation of FIGS. 2 and 3, “M” denotes the modules (M1 to M10), “t” denotes the control temperature of the corresponding module, “NV” represents the number of unit screw sections, “TV” represents the type of screw, “P/A” represents the screw pitch or alternatively the angle of the lobes and “L” represents the length of each unit screw section.

The extruder is made up of a succession of 10 modules (M1 to M10) of the same length, linearly linked together. The barrel of module M1 is open so as to allow solid lignocellulosic material to be introduced into the twin-screw extruder. The barrel of module M2 has a lateral orifice for introducing said aqueous composition into the extruder barrel and in contact with the lignocellulosic material being conveyed. In Example 1, said aqueous composition is water introduced into the extruder barrel by means of a pump at a flow rate of the order of 1.2 kg/h. The open barrel of module M6 is suitable for allowing the evacuation of water vapour produced upstream due to said thermomechanical treatment. The barrel of module M8 has an orifice communicating with a lateral member for introducing said fungal composition into said hydrated composition being conveyed in the twin-screw extruder, the introduction member comprising a piston pump (Milroyal® Dosapro, Milton Roy) delivering a flow rate of said fungal composition of 1.6 kg/h.

The twin-screw extruder is equipped, as described in FIG. 2, with:
conveyor screw sections, noted C2F, conjugated and double-threaded with a screw pitch of a length of 1.25D or 1D or 0.75D or 0.5D, representing the length of each screw section. In the example given, the constant “D” is equal to 25 mm. Such C2F type screw conveyors allow the lignocellulosic material to be conveyed longitudinally in the barrel, while at the same time allowing the lignocellulosic material to be blended due to the co-penetrating profile of the screws;
trapezoidal screw sections, noted as T1F, with a single thread and a screw pitch of 0.75D, suitable for allowing the lignocellulosic material to be introduced into the twin-screw extruder barrel and conveyed;
bilobal blending discs, noted as BB 90°, the bilobes of which are oriented perpendicular to the splined shafts and are offset from each other by an angle of 90°. Such blending discs are suitable for allowing the application of moderate mechanical stresses to the lignocellulosic material and efficient blending of the lignocellulosic fibres in the aqueous composition. Such blending discs also allow moderate shear forces to be applied to the solid fragments.

Fungal Composition

The fungal composition is formed from a mycelial suspension in sterile water of the filamentous fungus Grammothele fuligo previously grown in a bioreactor. The fungal composition has a dry matter content of 7.5 g/L of mycelial suspension.

Prior to the introduction of the shives into the twin-screw extruder, modules 6 to 10 of the extruder are treated with a steam jet to limit the risk of contamination by the extruder itself. Also, the pump of the device for introducing said fungal composition into said hydrated composition is treated with a 70° aqueous ethanol mixture.

The shives are introduced into the extruder at an introduction rate of 1 kg/h corresponding to an introduction rate expressed in terms of mass of dry matter of the shives of 0.87 kg/h.

Results

Samples of said composite material produced downstream of the extruder are taken up at the extruder outlet in filter culture bags designed for growing fungi on a solid substrate. An analysis of said composite material formed via the process according to the invention, by successive sieving on sieves with calibrated lattices, is given in Table 2 hereinbelow, in which “d” represents the diameter of the fragments and “L” represents their length.

TABLE 2

| | | | | |
|---|---|---|---|---|
| Size, mm | 8 > d > 4 | 4 > d > 2.4 | 2.4 > d > 1 | d < 1 mm |
| Mass proportion, % | 0 | 2.1 | 49.0 | 48.9 |
| Length, mm | — | 5 < L < 12 | 2 < L < 9 | L < 8 |

Thus, the following are obtained:
deaggregation of the lignocellulosic fibres of said shives as indicated by comparison of Tables 1 and 2;
inhibition of the endogenous flora of the shives. Such inhibition was analysed by spreading serial dilutions of the supernatant of a dispersion of said composite material in sterile water in standard agar and Sabouraud agar Petri dishes;
homogeneous inoculation of the filamentous fungi of said fungal composition leading to homogeneous growth of the filamentous fungus by fermentation in a solid medium (or support) (FMS).

By forming said composite material in a mould, followed by a step of fermenting said composite material on a solid support for 7 to 15 days at a temperature of 25° C. and producing a material enriched with mycelium, the process for preparing said composite material makes it possible to produce a solid object formed by a moulded material of low density, notably a density of the order of 0.09 g/cm³. However, the process for preparing said composite material also allows, after drying and, if necessary, grinding the mycelium-enriched material, the manufacture of thermopressed materials with increased mechanical strength due to the introduction of proteins and chitin resulting from the development of the filamentous fungus.

Needless to say, the invention is not limited to the use of shives and the density of the solid object formed from a moulded material may vary according to the density of the chosen solid lignocellulosic starting material and according to the forming conditions.

There is also nothing to prevent the use of a composite material according to the invention for the manufacture of a solid object formed from a thermopressed material and having a density of greater than 0.1 g/cm³, notably between 0.6 g/cm³ and 1.4 g/cm³, in particular between 0.6 g/cm³ and 1.0 g/cm³. Such a thermopressed material may have a flexural strength value of between 8 MPa and 40 MPa, notably between 9 MPa and 20 MPa, and a flexural modulus of elasticity of between 800 MPa and 6000 MPa, notably between 800 MPa and 2300 MPa.

### Example 2—Seeding of Shives with a Solid Composition of the Filamentous Fungus Lentinula edodes

The process for Example 2 is the same as for Example 1, using a Clextral EV25 twin screw extruder (Clextral SA, Firminy, France) represented schematically in FIG. 3. The barrel of the M8 module has an aperture communicating with a side member for introducing said fungal composition comprising mycelium of the filamentous fungus Lentinula edodes grown on a solid medium.

The extruder barrel represented schematically in FIG. 3 extends over a total length of 1000 mm. It is made up of a succession of 10 modules (M1 to M10) of the same length, linearly linked together. The barrel of module M1 is open so as to allow solid lignocellulosic material to be introduced into the twin-screw extruder. The barrel of module M2 has a lateral orifice for introducing said aqueous composition into the extruder barrel and in contact with the lignocellulosic material being conveyed. In Example 2, said aqueous composition is water introduced into the extruder barrel by means of a pump with a flow rate of the order of 1.1 kg/h. The barrel of module M6 has a lateral orifice for introducing water into the extruder barrel. In Example 2, a second injection of water into the barrel at module M6 is performed by means of a pump with a flow rate of about 1.95 kg/h. This addition of water makes it possible to control the water content of said composite material leaving the extruder and to allow the subsequent development of the filamentous fungus/fungi. The barrel of module M8 has an orifice communicating with a lateral member for introducing said fungal composition into said hydrated composition being conveyed in the twin-screw extruder, the introduction member comprising a piston pump delivering a flow rate of said fungal composition of 1.6 kg/h.

The twin-screw extruder is equipped, as described in FIG. 3, with:
conveying screw sections, noted C2F, conjugate and double-threaded with a screw pitch of a length of 1.25D or 1D or 0.75D or 0.5D, the constant “D” representing the length of each screw section. In the example given, the constant “D” is equal to 25 mm. Such C2F type screw conveyors allow the lignocellulosic material to be conveyed longitudinally in the barrel, while at the same time allowing the lignocellulosic material to be blended due to the co-penetrating profile of the screws;
trapezoidal screw sections, noted as T1F, with a single thread and a screw pitch of 0.75D, suitable for allowing the lignocellulosic material to be introduced into the twin-screw extruder barrel and conveyed;
bilobal blending discs, noted as BB 90°, the bilobes of which are oriented perpendicular to the splined shafts and are offset from each other by an angle of 90°. Such blending discs are suitable for allowing the application of moderate mechanical stresses to the lignocellulosic material and efficient blending of the lignocellulosic fibres in the aqueous composition. Such blending discs also allow moderate shear forces to be applied to the solid fragments;
bilobal blending discs, noted as BB+45°, the bilobes of which are oriented perpendicular to the splined shafts and are offset from each other by an angle of +45°. Such blending discs are suitable for allowing the application of less intense mechanical stresses than BB 90°. Such blending discs are chosen to allow efficient blending of the lignocellulosic fibres in the aqueous composition while at the same time ensuring that they are conveyed.

Thus, the following are obtained:
deaggregation of the lignocellulosic fibres of the shives as indicated by comparison of Tables 1 and 2;
partial inhibition of the endogenous flora of the shives;
homogeneous inoculation of the filamentous fungi of said fungal composition leading to homogeneous growth of the filamentous fungus by fermentation in a solid medium (or support) (FMS).

By forming said composite material in a mould, followed by a step of fermenting said composite material on a solid support for 7 to 15 days at a temperature of 25° C. and producing a material enriched with mycelium, the process for preparing said composite material makes it possible to produce a solid object formed by a moulded material of low density.

The invention may be the subject of numerous variants and applications other than those described hereinabove. In particular, it goes without saying that, unless otherwise indicated, the various structural and functional characteristics of each of the embodiments described hereinabove must not be considered as combined and/or strictly and/or inextricably linked to each other, but, on the contrary, as simple juxtapositions. In addition, the structural and/or functional characteristics of the various embodiments described hereinabove may form the subject totally or partly of any different juxtaposition or of any different combination.

1. A process for preparing a solid lignocellulosic material, referred to as a composite material, seeded with at least one organism, referred to as a filamentous fungus, which is a mycelium-forming multicellular eukaryote, said method comprising the steps of:
at least one solid lignocellulosic material impregnated with an aqueous composition is subjected to a treatment, known as a thermomechanical treatment, in which
said at least one impregnated lignocellulosic material is subjected to a succession of mechanical compression, expansion and shearing phases by blending at least one solid lignocellulosic material of said at least one impregnated lignocellulosic material, in contact with the aqueous composition;
said at least one impregnated lignocellulosic material is brought to a temperature above 50° C.;
whereby a composition, referred to as a hydrated composition, comprising a solid lignocellulosic material, referred to as hydrated lignocellulosic material, the specific surface area and moisture content of which are increased relative to the specific surface area and moisture content of said at least one starting lignocellulosic material, is formed as a result of this blending and this heating, said hydrated lignocellulosic material being suitable for being colonized by said at least one filamentous fungus, and then;
a composition, referred to as a fungal composition, comprising said filamentous fungus is added to said hydrated composition undergoing blending under blending conditions suitable for preserving the viability of at least one such filamentous fungus and for allowing subsequent development of said filamentous fungus in said composite material;
in which process the successive steps are performed continuously in at least one twin-screw extruder between an upstream inlet of said at least one twin-screw extruder, via which said at least one solid lignocellulosic material is introduced, and a downstream outlet of said at least one twin-screw extruder, via which said composite material is discharged.

2. The process according to claim 1, wherein said process is performed continuously by means of a single twin-screw extruder.

3. The process according to claim 1, wherein said thermomechanical treatment is a treatment for inactivating at least part of the endogenous microbial flora of said at least one solid lignocellulosic material.

4. The process according to claim 1, wherein said hydrated composition is cooled to a temperature below 50° C. prior to the addition of said fungal composition.

5. The process according to claim 1, wherein said at least one impregnated lignocellulosic material comprises an amount of said at least one solid lignocellulosic material such that the ratio of the mass of dry matter of said at least one solid lignocellulosic material to the mass of said at least one impregnated lignocellulosic material is between 30% and 60%.

6. The process according to claim 1, wherein said hydrated lignocellulosic material has a particle size less than that of said at least one solid lignocellulosic starting material.

7. The process according to claim 1, at least one filamentous fungus is chosen from the group formed by organisms of the phylum Basidiomycota, and in particular chosen from the group formed by Grammothele fuligo, Pleurotus citrinopileatus, Lentinula edodes, Pleurotus ostreatus, Pleurotus pulmonarius, Pleurotus columbinus, oyster mushroom hybrids, Ganoderma resinaceum, Agrocybe brasihensis, Flammulina velutipes, Hypholoma capnoides, Hypholoma sublaterium, Morchella angusticeps, Macrolepiota procera, Coprinus comatus, Agaricus arvensis, Ganoderma tsugae, Ganoderma lucidum and Inonotus obliquus.

8. The process according to claim 1, wherein said at least one solid lignocellulosic material comprises:
a mass proportion of celluloses, expressed as dry weight of celluloses and as dry weight of said at least one solid lignocellulosic material, of between 20% and 99%;
a mass proportion of hemicelluloses, expressed as dry weight of hemicelluloses and as dry weight of said at least one solid lignocellulosic material, of between 10% and 50%;
a mass proportion of lignins, expressed as dry weight of lignins and as dry weight of said at least one solid lignocellulosic material, of between 0.1% and 35%.

9. The process according to claim 1, wherein at least one solid lignocellulosic material is chosen from the group formed from all or part of a herbaceous plant, a cereal, a wheat, barley, rice or oat plant, a cereal straw, the stalks of a cultivated plant, of sorghum, corn or sugar cane stalks, all or part of a woody plant, of bark or wood chips, waste product from a plant resulting from the upgrading of said plant, shives, oilseed cake, all or part of a plant producing vegetable fibres, of sisal, flax, coconut, hemp, jute, ramie, cotton and nettle.

10. The process according to claim 1, wherein the heating temperature of said at least one impregnated lignocellulosic material during the thermomechanical treatment is between 50° C. and 180° C.

11. The process according to claim 1, wherein said fungal composition is added to said hydrated composition maintained at a temperature of between 10° C. and 30° C.

12. The process according to claim 1, wherein said thermomechanical treatment is performed continuously in at least one twin-screw extruder comprising, from upstream to downstream, a succession of rotary screw sections coupled to a tubular barrel of the twin-screw extruder, suitable for said at least one impregnated lignocellulosic material to be subjected, during its conveying from upstream to downstream in the twin-screw extruder, to increasing compression, shear and expansion stresses.

13. The process according to claim 12, wherein the succession of rotary screw sections coupled to the tubular barrel of the twin-screw extruder comprises, from upstream to downstream, in this order:
at least one section equipped with conveying screws chosen from the group formed by single-threaded conjugate screws of C1F type, double-threaded conjugate screws of C2F type, double-threaded trapezoidal screws of T2F type, single-threaded trapezoidal screws of T1F type and variants thereof and then
at least one section equipped with a stressing screw chosen from the group consisting of monolobal screws mounted at +45° and bilobal screws mounted at +45°, monolobal screws mounted at +90°, bilobal screws mounted at +90°, monolobal screws mounted at −45°, bilobal screws mounted at −45° and inverted screws, known as counter-threads, of the CF2C openwork type; and then
at least one section equipped with conveying screws chosen from the group formed by single-threaded conjugate screws of the C1F type, double-threaded conjugate screws of the C2F type, single-threaded trapezoidal screws of the T1F type, double-threaded trapezoidal screws of the T2F type and variants thereof.

14. The process according to claim 1, wherein said fungal composition is a liquid composition or a solid composition.

15. The process for manufacturing a solid object (11), in which use is made of a composite material (1) obtained via a process according to one of claims 1 to 14.

16. The process according to claim 15, wherein, the solid object being a low-density moulded solid object:
said composite material is formed; and then
the formed composite material is subjected to a step of fermentation, known as fermentation in a solid medium, and of development of said at least one filamentous fungus in said composite material, whereby a mycelium-enriched material is formed; and then
said mycelium-enriched material is dried so as to form the moulded solid object formed from a low-density composite material.

17. The process according to claim 15, wherein, the solid object being a solid object with a density of greater than 0.1 g/cm³:
said composite material is subjected to a step of fermentation (12), known as fermentation in a solid medium, and of development of said at least one filamentous fungus in said composite material, whereby a mycelium-enriched material is formed; and then
said mycelium-enriched material is subjected to a step of forming by thermocompression so as to form the solid object with a density of greater than 0.1 g/cm³.

18. A solid lignocellulosic material (1), referred to as a composite material, comprising lignocellulosic fibres and at least one organism (2), referred to as a filamentous fungus, which is a mycelium-forming multicellular eukaryote, said composite material (1) being predominantly in the form of particles of generally elongate shape and having a largest dimension greater than each of the two dimensions orthogonal to the largest dimension and orthogonal to each other, the ratio of the largest dimension to each of the dimensions orthogonal to the largest dimension being greater than 2.

19. (canceled)

20. (canceled)
